(19) United States
(12) Patent Application Publication
(10) Pub. No.: US 2024/0316314 A1
(43) Pub. Date: Sep. 26, 2024

(54) CATHETER

(71) Applicant: KANEKA CORPORATION, Osaka-shi, Osaka (JP)

(72) Inventor: Toshiya KISA, Okaya-shi (JP)

(73) Assignee: KANEKA CORPORATION, Osaka-shi, Osaka (JP)

(21) Appl. No.: 18/579,716

(22) PCT Filed: May 17, 2022
(86) PCT No.: PCT/JP2022/020555

§ 371 Date: Jan. 16, 2024

(30) Foreign Application Priority Data
Aug. 19, 2021 (JP) 2021-134329

(51) Int. Cl. A61M 25/00

(52) U.S. Cl. CPC A61M 25/005

(57) ABSTRACT

A catheter has a shaft, a coil, a leaf spring having a first connection part where its proximal end part is connected to the distal end part of the coil, at least one wire, and a tubular member disposed outside the coil such that the wire, the leaf spring, and the first connection part are located in the lumen, wherein the tubular member has a first section from the distal end of the coil to a position D having a length d proximally, an inner wall of the tubular member is not in contact with an outer surface of the coil in the first section, and the length d is a length of one winding of the coil or longer and a length of 30 windings or shorter. A catheter can easily achieve the desired curved shape by preventing twisting and kinking of the catheter distal part.

### TECHNICAL FIELD

The present invention relates to a catheter whose distal portion is bendable.

### BACKGROUND ART

A catheter that is inserted into the inside of the heart through arteries is used during the examination and treatment of cardiac diseases. The distal part of the catheter needs to be bent to follow blood vessels in the heart in order to deliver the tip of the catheter to the desired location in the heart, and a catheter that allows the distal part of the catheter to be curved by operating a handle placed at the proximal end side of the catheter has been developed. The catheter whose distal portion is bendable usually allows the distal portion to be curved by operating a handle to pull a wire fixed inside the tip of the catheter.

As such a catheter, a catheter has been proposed in which the distal end of a leaf spring, which is a member that bends the distal part of the catheter, and the distal end of the wire are fixed to the distal end of the catheter, and the proximal end of the leaf spring is connected to a tube located in the lumen of the catheter.

By the way, in order to deliver the tip of the catheter to the desired site according to the size of the heart or the purpose, it is required for the distal part of the catheter to have the ability to bend freely in the desired direction by wire operation to follow tortuous vessels (trackability). In addition, to prevent the distal part of the catheter from traveling in an undesired direction and damaging the wall of the lumen of the living body, and to improve the ability of forces by the operation from the proximal side to be transmitted to the distal part of the catheter (pushability), defects such as twisting and bending (kinking) need to be suppressed when the distal part of the catheter is curved. Accordingly, various forms of fixation of the proximal side of the leaf spring, which is a member that is placed in the distal part of the catheter and bends the distal part of the catheter, have been proposed.

In the catheters disclosed in Patent documents 1 and 2, a wire is disposed in a wire tube, and the proximal end of a leaf spring is fixed to a coil tube. In the catheter disclosed in the Patent document 2, the wire tube is fixed to the surface of the leaf spring via an adhesive layer. In Patent document 3, a catheter in which a leaf spring is fixed to a coil tube via a coil stopper is disclosed. In the catheter disclosed in Patent document 4, a wire and a leaf spring are disposed in a distal tube, and the proximal end of the leaf spring is fixed to a proximal tube. In addition, it is disclosed that a protection tube is disposed outside the proximal tube, and the proximal end of the distal tube is located distal to the distal end of the protection tube.

### RELATED ART DOCUMENT

### Patent Document

Patent document 1: JP 2006-61350 A
Patent document 2: JP 2012-200445 A
Patent document 3: JP 2014-64614 A
Patent document 4: WO 2019/156059

### SUMMARY OF THE INVENTION

### Problems to be Solved by the Invention

However, in conventional catheters, when a coil connected to the proximal end part of the leaf spring is provided to ensure stiffness at the base of the bend, the coil is compressed by pulling the wire and deformed to bulge in the radial direction, causing the leaf spring connected to the coil to rotate, which leads to the problem that the distal part of the catheter bends torsionally. To prevent this, the connection part between the coil and the leaf spring can be fixed to the catheter tube, but the fixed part becomes rigid, resulting in a stiffness step with a sudden change in stiffness in the longitudinal axis direction of the catheter tube. Therefore, when the distal part of the catheter is bent, the distal part of the catheter may break, which is a kink, at the point where the stiffness step is generated, and there was room for improvement. Furthermore, there was room for improvement in that the wire, which is a member for pulling, and the leaf spring, which is a member for bending, were interfered with by other members, adversely affecting the curved shape of the distal part of the catheter.

The interference of other members with the member for pulling or member for bending when the distal part of the catheter is curved, as well as twisting or kinking of the distal part of the catheter may lead to reduced pushability, resulting in a catheter with reduced trackability because the desired curved shape cannot be obtained. The present invention has been made in view of the above, and the purpose thereof is to provide a catheter that can easily achieve the desired curved shape by preventing twisting and kinking of the distal part of the catheter when being curved, while preventing interference of the tubular member with the wire as a member for pulling and the leaf spring as a member for bending when the distal part of the catheter is curved.

### Means for Solving the Problems

A catheter that can solve the above problems has a shaft having a distal end and a proximal end in a longitudinal axis direction and having a lumen extending in the longitudinal axis direction, a coil having a lumen extending in the longitudinal axis direction and disposed in the lumen of the shaft; a leaf spring extending in the lumen of the shaft and having a first connection part where a proximal end part of the leaf spring is directly or indirectly connected to a distal end part of the coil and a second connection part where a distal end of the leaf spring is directly or indirectly connected to a distal end part of the shaft; at least one wire extending in the lumen of the coil, a distal side of the wire directly or indirectly connected to the distal end part of the shaft; and a tubular member having a lumen extending in the longitudinal axis direction and disposed outside the coil such that the wire, the leaf spring, and the first connection part are located in the lumen of the tubular member, wherein, in the longitudinal axis direction, the tubular member has a first section from a distal end of the coil to a position D having a length d proximally and a second section from the position D to a proximal end of the tubular member, an inner wall of the tubular member is not in contact with an outer surface of the coil in the first section, and the length d is a length of one winding of the coil or longer and a length of 30 windings of the coil or shorter. Such a configuration can prevent the tubular member from interfering with the wire that is pulled for bending the distal part of the catheter and the leaf spring that forms the curving shape of the distal part of the catheter when the distal part of the catheter is curved. This makes it easier for the force from the hand side to be transmitted to the wire located at the distal part of the catheter, allowing the curving of the leaf spring by pulling the wire to be less susceptible to the tubular member, and the catheter to be less prone to kinking. In addition, this can prevent the coil from deforming to bulge in the radial direction even if the coil is compressed along with the traction of the wire, preventing the curving of the distal part of the catheter from kinking caused by the rotation of the leaf spring connected to the coil. Since how easy it is for the coil to bulge depends on the number of windings of the coil, the above effect can be achieved by the length of the first section in the longitudinal axis direction being a length of the predetermined number of windings of the coil or shorter. In addition, the distal part of the catheter can prevent kinking because it is less likely to have stiffness steps in the longitudinal axis direction of the distal part of the catheter. As a result, it becomes easier for the distal part of the catheter to have a desired curved shape.

Preferably, a part of the inner wall of the tubular member is in contact with the outer surface of the coil in the second section.

Preferably, the first connection part is located in the first section in the longitudinal axis direction.

Preferably, the tubular member extends to the second connection part, and the leaf spring and the wire are disposed in the lumen of the tubular member in the distal end part of the shaft.

Preferably, the proximal end of the tubular member is located distal to a proximal end of the coil, and the coil has a first fixed part fixed to the shaft at a position proximal to the proximal end of the tubular member in the longitudinal axis direction. In this case, preferably, a length from a distal end of the first fixed part to the proximal end of the tubular member is a length of 20 windings of the coil or shorter in the longitudinal axis direction.

Preferably, the tubular member has a second fixed part fixed to the shaft in the second section. In this case, preferably, a length from a distal end of the second fixed part to the distal end of the coil is twice a length of the leaf spring or shorter in the longitudinal axis direction. Furthermore, in this case, in the longitudinal axis direction, a distal end of the second fixed part is located proximal to a proximal end of the first connection part, and a length from the distal end of the second fixed part to the proximal end of the first connection part is ¼ a length of the leaf spring or longer.

### Effects of the Invention

According to the present invention, in the catheter whose distal part is bendable, the tubular member can be prevented from interfering with the wire and the leaf spring when the distal part of the catheter is curved. This makes it easier for the force from the hand side to be transmitted to the wire located at the distal part of the catheter, resulting in the catheter that allows the curving of the leaf spring by pulling the wire to be less susceptible to the tubular member. In addition, the distal part of the catheter can be prevented from twisting and kinking. As a result, a desired curved shape of the distal part of the catheter can be achieved by the wire operation, and the catheter that can be easily delivered to a desired location can be provided.

### BRIEF DESCRIPTION OF THE DRAWINGS

FIG. 1 is a plan view of a catheter in accordance with one embodiment of the present invention.

FIG. 2 is a cross-sectional view of the distal part of the catheter shown in FIG. 1 in the longitudinal axis direction (partially a plan view).

FIG. 3 is a cross-sectional view of the catheter along the III-III line of FIG. 2.

FIG. 4 is a cross-sectional view of the catheter along the IV-IV line of FIG. 2.

FIG. 5 is a cross-sectional view of the catheter along the V-V line of FIG. 2.

FIG. 6 is a cross-sectional view of the catheter along the VI-VI line of FIG. 2.

FIG. 7 is a plan view of a coil in accordance with one embodiment of the present invention.

FIG. 8 is a plan view of the coil shown in FIG. 7 at maximum compression.

FIG. 9 is a cross-sectional view of a distal part of a catheter in accordance with another embodiment of the present invention in the longitudinal axis direction (partially a plan view).

FIG. 10 is a cross-sectional view of a distal part of a catheter in accordance with still another embodiment of the present invention in the longitudinal axis direction (partially a plan view).

FIG. 11 is a cross-sectional view of the catheter along the XI-XI line of FIG. 10.

FIG. 12 is another example of the cross-sectional view shown in FIG. 11.

FIG. 13 is a cross-sectional view of a distal part of a catheter in accordance with still another embodiment of the present invention in the longitudinal axis direction (partially a plan view).

FIG. 14 is a cross-sectional view of the catheter along the XIV-XIV line of FIG. 13.

FIG. 15 is another example of the cross-sectional view shown in FIG. 14.

### MODE FOR CARRYING OUT THE INVENTION

Hereinafter, the present invention will be described based on the following embodiments, however, the present invention is not limited by the following embodiments and can be altered in design within a scope in compliance with the intent described above and below, and all the changes are to be encompassed within a technical scope of the present invention. Note that, in each drawing, hatching, reference signs for components, and the like may be omitted for convenience of description, and in such a case, the specification and other drawings are to be referred to. Furthermore, since the dimensions of the various components in the drawings are provided for the purpose of facilitating the understanding of the feature of the present invention, the dimensions may differ from the actual dimensions in some cases.

A catheter in accordance with an embodiment of the present invention has a shaft having a distal end and a proximal end in a longitudinal axis direction and having a lumen extending in the longitudinal axis direction; a coil having a lumen extending in the longitudinal axis direction and disposed in the lumen of the shaft; a leaf spring extending in the lumen of the shaft and having a first connection part where a proximal end part of the leaf spring is directly or indirectly connected to a distal end part of the coil and a second connection part where a distal end part of the leaf spring is directly or indirectly connected to a distal end part of the shaft; at least one wire extending in the lumen of the coil, a distal side of the wire directly or indirectly connected to the distal end part of the shaft; and a tubular member having a lumen extending in the longitudinal axis direction and disposed outside the coil such that the wire, the leaf spring, and the first connection part are located in the lumen of the tubular member, wherein, in the longitudinal axis direction, the tubular member has a first section from a distal end of the coil to a position D having a length d proximally and a second section from the position D to a proximal end of the tubular member, an inner wall of the tubular member is not in contact with an outer surface of the coil in the first section, and the length d is a length of one winding of the coil or longer and a length of 30 windings of the coil or shorter.

The catheter in accordance with embodiments of the present invention having the above configuration allows the distal part of the catheter located distal to the distal end part of the coil to be curved by curving the leaf spring connected to the distal end part of the shaft by pulling the wire while securing the stiffness of the base side of the distal side of the catheter distal to the coil. From the viewpoint of action, the wire can be referred to as a pulling member and the leaf spring can be referred to as a curving member. In the first section, the inner wall of the tubular member is not in contact with the outer surface of the coil, which can prevent interference of the tubular member with the wire, which is a pulling member, and the leaf spring, which is a curving member. This makes it easier for the force from the hand side to be transmitted to the wire located at the distal part of the catheter, resulting in the catheter that allows the curving of the leaf spring by pulling the wire to be less susceptible to the tubular member. In addition, the tubular member is disposed outside the coil, and the length of the first section in the longitudinal axis direction where the inner wall of the tubular member is not in contact with the outer surface of the coil is a length of 30 windings of the coil or shorter, allowing a part of the tubular member near the distal end of the coil, which is the base of the curving, to be in contact with the outer surface of the coil, and thus preventing the coil from deforming to bulge in the radial direction even if the coil is compressed along with the traction of the wire, preventing the curving of the distal part of the catheter from kinking caused by rotation of the leaf spring connected to the coil. Since how easy it is for the coil to bulge depends on the number of windings of the coil, the above effect can be achieved by the length of the first section in the longitudinal axis direction being a length of the predetermined number of windings of the coil or shorter. Furthermore, the length of the first section in the longitudinal axis direction where the inner wall of the lumen of the tubular member is not in contact with the outer surface of the coil is a length of one winding of the coil or longer, preventing a stiffness step in the longitudinal axis direction of the distal part of the catheter, and preventing kinking when the distal part of the catheter is curved. As a result, a desired curved shape of the distal part of the catheter can be achieved to make the catheter capable of easily delivering the distal part of the catheter to a desired location, facilitating examination and treatment with the catheter and preventing accidents such as the distal part of the catheter damaging the lumen wall of the living body or traveling in an undesired direction.

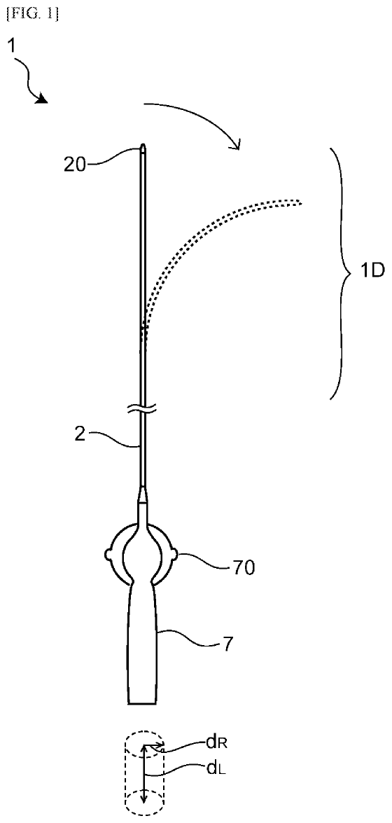

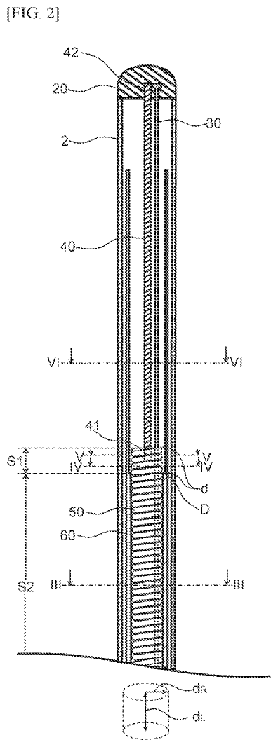

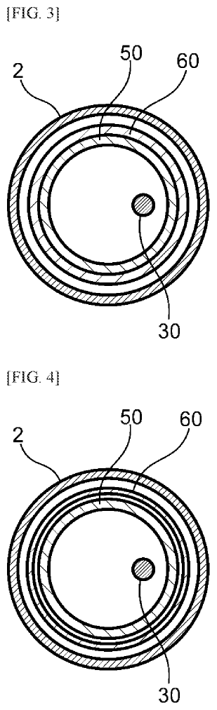

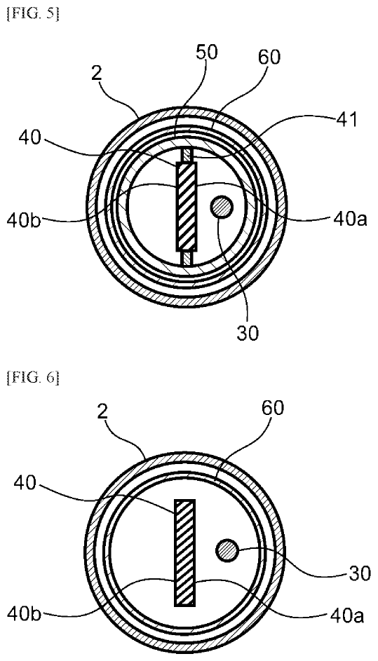

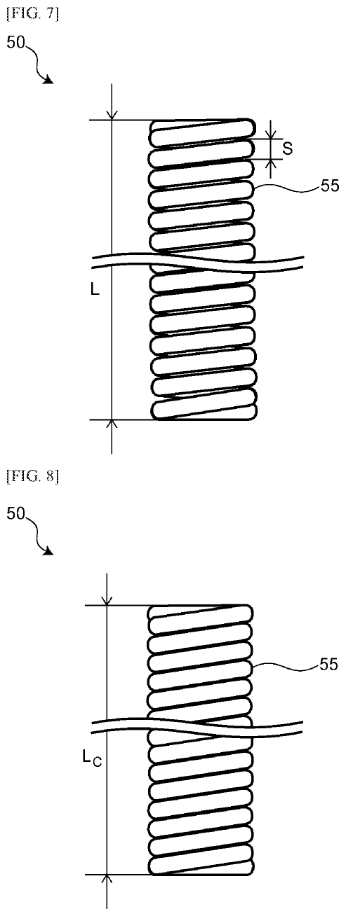

Referring to FIG. 1 to FIG. 8, the catheter in accordance with embodiments of the present invention will be described. FIG. 1 is a plan view of a catheter in accordance with one embodiment of the present invention, and the dotted line shows how the distal part of the catheter is curved on one side of the leaf spring in the radial direction of the shaft. FIG. 2 is a cross-sectional view of the distal part of the catheter shown in FIG. 1 in the longitudinal axis direction when the distal part of the catheter is not curved, and the coil is shown as a plan view. FIG. 3 to FIG. 6 are cross-sectional views of the catheter shown in FIG. 2 at different positions in the longitudinal axis direction, respectively, and FIG. 3 is a cross-sectional view along the III-III line of FIG. 2, FIG. 4 is a cross-sectional view along the IV-IV line of FIG. 2, FIG. 5 is a cross-sectional view along the V-V line of FIG. 2, and FIG. 6 is a cross-sectional view along the VI-VI line of FIG. 2. FIG. 7 is a plan view of a coil in accordance with one embodiment of the present invention, and FIG. 8 is a plan view of the coil shown in FIG. 7 at maximum compression.

In the present invention, a proximal side refers to the direction towards a user's hand in the extending direction of a catheter 1, and a distal side refers to the opposite side of the proximal side, that is, the direction towards the person to be treated. The extending direction of the catheter 1 is preferably the same as a longitudinal axis direction d_{L} of the shaft 2. In a cross section perpendicular to the longitudinal axis direction d_{L}, the direction connecting the center of a shaft 2 and a point on the circumscribed circle of the shaft 2 refers to a radial direction d_{R}. In the figures in the present description, the lower side of the figures is the proximal side and the upper side of the figure is the distal side.

As shown in FIG. 1, the catheter 1 has the shaft 2 having a distal end and a proximal end in the longitudinal axis direction d_{L} and having a lumen extending in the longitudinal axis direction d_{L}. The distal end side of the shaft 2 is inserted into the living body, and the distal end part is delivered to a treatment site by operating the proximal end side. Accordingly, flexibility is preferred, and metals and resins can be used as a material. Since the shaft 2 is inserted into the living body, it is preferably made of biocompatible materials.

Materials forming the shaft 2 exemplified by synthetic resin such as polyolefin-based resin such as polyethylene and polypropylene; polyamide-based resin such as nylon; polyester-based resin such as PET; polyimide-based resin; aromatic polyetherketone-based resin such as PEEK; polyetherpolyamide-based resin; polyurethane-based resin; fluorine-based resin such as PTFE, PFA, and ETFE; polyvinyl chloride-based resin; silicone-based resin, and natural rubber. The shaft 2 may have a single-layer or multi-layer structure.

When the shaft 2 is multi-layered, for example, the intermediate layer of a resin tube constituting the shaft 2 may be formed with a metal braid such as stainless steel, carbon steel, nickel-titanium alloy, and the like.

Electrodes, sensors, and other components for examination and treatment can be placed on the surface of the shaft 2. The lumen of the shaft 2 can be used to place internal structures for curving a catheter distal part 1D, conductive wires connected to the electrodes and sensors, and other components. The distal side of the conductive wires are connected to the electrodes disposed on the surface of the shaft 2, and the proximal side of the conductive wires are connected to a detector or a power source through the proximal side of the catheter 1, thereby electronic signal can be received, and the electrodes can be energized. The catheter 1 having such a configuration can be used as an electrode catheter that measures cardiac potentials or an ablation catheter cauterizes tissue. The length of the shaft 2 in the longitudinal axis direction d_{L}, the outer diameter, the thickness, and the like can be appropriately selected according to the intended use.

A tip part 20 is preferably disposed at the distal end part of the shaft 2. The tip part 20 may be a different member from the shaft 2, or may be formed as a part of the same member. When the tip part 20 is a different member from the shaft 2, the tip part 20 may have a part that is inserted into the lumen of the shaft 2 or a part that protrudes distally from the distal end of the shaft 2. When the tip part 20 is formed as a part of the same member as the shaft 2, the tip part 20 may be formed by thermally fused the distal end part of the shaft 2 or otherwise sealed to close the opening at the distal end of the shaft 2.

A handle 7 is preferably disposed at the proximal side of the shaft 2, and the proximal end of the shaft 2 is preferably fixed to the inside of the handle 7. In the handle 7, proximal end parts of conductive wires extending from the shaft 2 or a wire 30 described below are preferably disposed. To easily operate the wire 30, the handle 7 may be provided with a wire operation part 70. By fixing the proximal end part of the wire 30 to the wire operation part 70, operating the wire operation part 70 to pull or release the wire 30 can bend or undo the catheter distal part 1D.

As shown in FIG. 2, the catheter 1 has the wire 30 extending in the lumen of the shaft 2, a leaf spring 40, and a coil 50 having a lumen extending in the longitudinal axis direction d_{L}. The wire 30 is a pulling member, the leaf spring 40 is a curving member, and the coil 50 is a member located at the base side of the curving. The leaf spring 40 is curved by pulling the wire 30, and the catheter distal part 1D distal to the distal end part of the coil 50 can be curved.

As shown in FIG. 2, FIG. 5, and FIG. 6, preferably, the leaf spring 40 extends in the longitudinal axis direction d_{L}, and has a plate-like shape having one surface 40a and the other surface 40b. The leaf spring 40 having a plate-like shape can define the direction of curvature of the catheter distal part 1D and allow the catheter distal part 1D to be curved towards the one surface 40a side and/or the other surface 40b side. The leaf spring 40 has a first connection part 41 where the proximal end part of the leaf spring 40 is directly or indirectly connected to the distal end part of the coil 50. The proximal end part of the leaf spring 40 may be fixed to the distal end of the coil 50 by adhesive, solder, laser welding, or the like to form the first connection part 41 as shown in FIG. 5. Alternatively, although not shown in the figures, the proximal end part of the leaf spring 40 may be brought into contact with the distal end part of the coil 50 to form the first connection part 41 without any particular fixing means. The first connection part 41, where the proximal end part of the leaf spring 40 and the distal end part of the coil 50 are connected, may be formed indirectly by contacting or fixing the proximity of the respecting ends without the respective ends being directly contacted or fixed.

The positional relationship of the leaf spring 40 and the coil 50 in the first connection part 41 may be any, for example, the proximal end of the leaf spring 40 may be positioned in the lumen of the coil 50 or outside the coil 50. Alternatively, the proximal end of the leaf spring 40 may be in contact with or fixed to the distal end of the coil 50. Particularly, among such positional relationships, a part of the proximal end part of the leaf spring 40 is preferably disposed in the lumen of the coil 50 at the first connection part 41, with the coil 50 receiving the proximal end part of the leaf spring 40. This allows the leaf spring 40 and the coil 50 to be stably connected.

The leaf spring 40 also have a second connection part 42 where the distal end part of the leaf spring 40 is directly or indirectly fixed to the distal end part of the shaft 2. At the second connection part 42, the distal end part of the leaf spring 40 may be directly fixed to the distal end part of the shaft 2. Alternatively, the second connection part 42 may be formed by fixing the proximity of the respective ends of the leaf spring 40 and the shaft 2 without the respective ends being directly fixed. For example, the distal end part of the leaf spring 40 may be indirectly fixed to the distal end part of the shaft 2 in such a way that the tip part 20 is provided at the distal end of the shaft 2 and the distal end part of the leaf spring 40 is fixed to the tip part 20. The method of fixing the distal end part of the leaf spring 40 and the distal end part of the shaft 2 is not particularly limited, and the method can be exemplified by adhesives, solder, or laser welding.

The leaf spring 40 is a spring made of a plate material, and the materials constituting the leaf spring 40 include, for example, stainless steel, titanium, carbon steel, nickel-titanium alloys, cobalt-chrome alloys, tungsten alloys, and other metals. Alternatively, the materials constituting the leaf spring 40 may be synthetic resin such as aromatic polyetherketone (for example, PEEK), polycarbonate resin, and fiber reinforced resins. Still, alternatively, the leaf spring 40 may be made of synthetic rubber such as butadiene rubber, isoprene rubber, styrene butadiene rubber, ethylene propylene rubber, acrylic rubber, and silicone rubber; and natural rubber. Of these, the material constituting the leaf spring 40 is preferably metal, more preferably stainless steel.

The wire 30 is a pulling member to manipulate the catheter distal part 1D in a curved manner. Pulling the wire 30 towards the proximal side makes the leaf spring 40 curved towards the one surface 40a side or the other surface 40b side, and thus, the catheter distal part 1D can be curved towards the one surface 40a side or the other surface 40b side. The degree of curvature of the leaf spring 40 can be adjusted by increasing or decreasing the force to pull the wire 30 towards the proximal side, and when the traction of the wire 30 is released, the curvature of the leaf spring 40 returns to its original state and the catheter distal part 1D can be returned to its original state before curving. For example, the wire 30 disposed at the one surface 40a side of the leaf spring 40 can make the leaf spring 40 curved towards the one surface 40a side; the wire 30 disposed at the other surface 40b side of the leaf spring 40 can make the leaf spring 40 curved towards the other surface 40b side. The wire 30 may be a plurality of wires, and for example, disposing each wire 30 on both sides of the one surface 40a side and the other surface 40b side of the leaf spring 40 can make the catheter 1 bendable towards both sides of the one surface 40a side and the other surface 40b side.

The wire 30 extends in the lumen of the coil 50 as shown in FIG. 2. The distal side of the wire 30 is directly or indirectly connected to the distal end part of the shaft 2 at a position distal to the distal end of the coil 50. This allows the catheter distal part 1D to be curved by pulling the wire 30. Of the distal side of the wire 30, the distal end part of the wire 30 is preferably connected to the distal end part of the shaft 2, and the distal end of the wire 30 may be connected to the distal end part of the shaft 2. The distal side of the wire 30 may be connected by directly being fixed to the distal end part of the shaft 2, or may be indirectly connected to the distal end part of the shaft 2 by being fixed to the tip part 20 or other member disposed at the distal end part of the shaft 2, for example the leaf spring 40 and the like. Which part of the distal end of the wire 30 is connected to the distal end part of the shaft 2, and to which part of the distal end part of the shaft 2 the wire 30 is connected can be appropriately selected according to the desired curving shape of the catheter distal part 1D.

As the wire 30, metal wire material such as stainless steel or material formed from synthetic resin such as fluorine resin can be used, and one wire 30 may be a single wire or have a structure consisting of multiple wires. The wire 30 and the shaft 2 may be connected by fixing in the same manner as described for fixing the leaf spring 40 and the shaft 2, or the distal end part of the wire 30 may be connected without any particular fixing means by contacting to thrust the distal end part of the shaft 2 or its proximity.

The coil 50 provided in the catheter 1 can ensure the stiffness of the base side of the curvature of the catheter distal part 1D, allowing the catheter distal part 1D at the side distal to the distal end part of the coil 50 in the longitudinal axis direction d_{L} to be curved. In order to achieve a certain degree of stiffness at the base side of the curvature of the catheter distal part 1D, the coil 50 is preferably anon-compressible, so-called tightly wound coil. When the coil 50 is a non-compressible, it is easier for the catheter distal part 1D to be curved starting from the distal end part of the coil 50. As shown in FIG. 7 and FIG. 8, strictly speaking, a non-compressible coil is the one having the ratio L_{c}/L of the total length L of the coil in its natural state with no external force applied to the coil and the total length L_{c} of the coil at maximum compression is 1, but a non-compressible coil also substantially includes a coil having a L_{c}/L of 0.95 or greater and a coil having a L_{c}/L of 0.9 or greater. The coil 50 that is such a non-compressible coil can easily ensure stiffness of the base side of the curvature of the catheter distal part 1D, allowing the catheter distal part 1D at the position distal to the distal end part of the coil 50 to be curved. The coil 50 may extend to the proximal end of the shaft 2, the coil 50 may extend beyond the proximal end of the shaft 2, or the proximal end of the coil 50 may be located midway of the shaft 2. The coil 50 may switch to a different tube in the middle of the shaft 2 in the longitudinal axis direction d_{L}.

The coil 50 preferably has flexibility and can be made of metal or resin as a material, for example metal wire such as stainless steel and nickel-titanium alloys, or synthetic resin wire such as aromatic polyetherketone resin (for example, PEEK) and polycarbonate resin. The cross-sectional shape of a coil wire 55 forming the coil 50 may be circular, square, or a combination thereof. Particularly, the coil 50 is preferably a coil wound with metal wire, and the coil 50 is more preferably a coil wound with the coil wire 55 made of stainless steel and circular in cross-sectional shape. When the coil 50 is formed by the coil wire 55 the cross-sectional shape of which is circular, the diameter of the coil wire 55 is preferably 0.1 mm to 0.5 mm. When the coil 50 is formed by the coil wire 55 the cross-sectional shape of which is square, the side of the square may be 0.05 mm to 1 mm. Since the internal structure of the catheter 1 in accordance with embodiments of the present invention switches at the distal end of the coil 50 in the longitudinal axis direction di, the material, size, and flexibility of the coil 50 are appropriately selected so that the stiffness of the catheter 1 does not differ too much between the distal and proximal sides to the distal end of the coil 50.

As shown in FIG. 2 and FIG. 5, the catheter 1 has a tubular member 60 having a lumen extending in the longitudinal axis direction d_{L}, and the tubular member 60 is disposed outside the coil 50 such that the wire 30, the leaf spring 40, and the first connection part 41 are located in the lumen of the tubular member 60. In the longitudinal axis direction d_{L}, the distal end of the tubular member 60 may be located at the proximity of the distal end part of the shaft 2 so that most of the leaf spring 40 is located in the lumen of the tubular member 60, or it may be located midway between the first connection part 41 and the distal end part of the shaft 2 so that a part of the leaf spring 40 is located in the lumen of the tubular member 60. The tubular member 60 may extend to the proximal end of the shaft 2, extend beyond the proximal end of the shaft 2, or the proximal end of the tubular member 60 may be located midway the shaft 2.

As the materials constituting the tubular member 60, reference can be made to the description of synthetic resins among the materials constituting the shaft 2, and among them, elastomers of polyolefin-based resin, polyimide-based resin, and polyamide-based resin are preferable.

As shown in FIG. 2, in the longitudinal axis direction d_{L}, the tubular member 60 has a first section S1 from the distal end of the coil 50 to a position D having a length d proximally and a second section S2 from the position D to the proximal end of the tubular member 60, an inner wall of the tubular member 60 is not in contact with an outer surface of the coil 50 in the first section S1, and the length d is a length of one winding of the coil 50 or longer and a length of 30 windings of the coil 50 or shorter. In other words, the catheter 1 in accordance with embodiments of the present invention has a configuration in which the tubular member 60 is disposed outside the coil 50 as shown in FIG. 3, the inner wall of the tubular member 60 is not in contact with the outer surface of the coil 50 in the first section S1 as shown in FIG. 4 and FIG. 5, and the leaf spring 40 and the wire 30 are disposed in the lumen of the tubular member 60 at the side distal to the distal end of the coil 50 as shown in FIG. 6. This allows the configuration to prevent interference of the tubular member 60 against the wire 30, which is a pulling member, and the leaf spring 40, which is a curving member. As a result, the force from the hand side is easily transmitted to the wire 30 located at the catheter distal part 1D, and the curvature of the leaf spring 40 by pulling the wire 30 is less susceptible to the tubular member 60, making it easier for the catheter distal part 1D to achieve a desired curving shape.

The tubular member 60 may extends to the proximal end of the coil 50 or the proximal end of the tubular member 60 may be located distal to the proximal end of the coil 50 in the longitudinal axis direction di. The tubular member 60 may consist of one member from the distal end to the proximal end, or may switch to another member at any position in the longitudinal axis direction d_{L}. The position at which the tubular member 60 switches to another member is not particularly limited, and for example, it may switch at the position D or it may switch at any other position. The tubular member 60 is preferably disposed outside the coil 50 in the section of 50% or longer of the length of the coil 50 in the longitudinal axis direction d_{L}. The section where the tubular member 60 is disposed outside the coil 50 is more preferably 60% or longer of the length of the coil 50, even more preferably 75% or longer, and may be 80% or longer, 90% or longer, or even 100%. With the tubular member 60 disposed outside the coil 50 in the above range, it is easier to prevent the coil 50 from bulging in the radial direction d_{R} thanks to the tubular member 60.

As shown in FIG. 2 to FIG. 5, the tubular member 60 disposed outside the coil 50 so that the first connection part 41 is located in its lumen, which prevents the coil 50 from being deformed to bulge in the radial direction d_{R} even if the coil 50 is compressed as the wire 30 is pulled when the catheter distal part 1D is curved, and prevents the curvature of the leaf spring 40 from kinking due to rotation of the first connection part 41. As a result, it becomes easier for the catheter distal part 1D to be curved into a desired shape.

Note that the length of one winding of the coil 50 is, as shown in FIG. 7, a length of one winding of the coil wire 55 forming the coil 50 in the longitudinal axis direction d_{L} of the coil 50 in its natural state with no external force applied to the coil 50. When the coil 50 is a non-compressible coil and the ratio L_{c}/L is 1 where L is the total length of the coil in its natural state and L is the total length of the coil at maximum compression, the length of one winding of the coil 50 is the same as the wire diameter of the coil wire 55 forming the coil 50. When the coil 50 is not completely a non-compressible coil, as shown in FIG. 7, the length of one winding of the coil 50 can be determined as a length S from one end of one winding to one end of the next winding of the coil wire 55 in the longitudinal axis direction d_{L} of the coil 50. When the coil 50 is not a non-compressible coil, the length S is the combined length of the wire diameter of the coil wire 55 and the length of the gap between the coil wires 55, which is the same as the pitch of the coil 50. For example, the length of 30 windings of the coil 50 is 30 times the length S.

The length d of the first section S1 of the tubular member 60 is a length of one winding or longer, preferably a length of 3 windings or longer, more preferably a length of 4 windings or longer, even more preferably a length of 5 windings or longer, and may be a length of 7 windings or longer. The inner wall of the tubular member 60 is not in contact with the outer surface of the coil 50 in the first section S1 having the length d the lower limit of which is in the above range, preventing interference of the tubular member 60 against the wire, which is a pulling member, and the leaf spring, which is a curving member. This allows the force from the hand side to be easily transmitted to the wire 30 located in the catheter distal part 1D, resulting in the catheter 1 that allows the curving of the leaf spring 40 by pulling the wire 30 to be less susceptible to the tubular member 60. In addition, since the lower limit of the length d of the first section S1 where the inner wall of the tubular member 60 is not in contact with the outer surface of the coil 50 at the distal end part of the coil 50 where the stiffness step occurs is in the above range, further stiffness step caused by the tubular member 60 can be suppressed, thus preventing kinking when the catheter distal part 1D is curved.

The length d of the first section S1 of the tubular member 60 is a length of 30 windings or shorter, preferably a length of 25 windings or shorter, more preferably a length of 20 windings or shorter, and may be a length of 15 windings or shorter, a length of 12 windings or shorter, or a length of 10 windings or shorter. In the second section S2 proximal to the first section S1 having the upper limit of the length d in the above range, the tubular member 60 may be brought into contact with the outer surface of the coil 50 until near the distal end of the coil 50, which is the base end of the curvature, so that the coil 50 can be prevented from deforming to bulge in the radial direction d_{R} even if it is compressed along with the traction of the wire 30, preventing the curving of the catheter distal part 1D from kinking caused by the rotation of the leaf spring 40 connected to the coil 50. Since how easy it is for the coil 50 to bulge depends on the number of windings of the coil 50, the above effect can be achieved by the length d of the first section S1 being a length of the predetermined number of windings of the coil 50 or shorter.

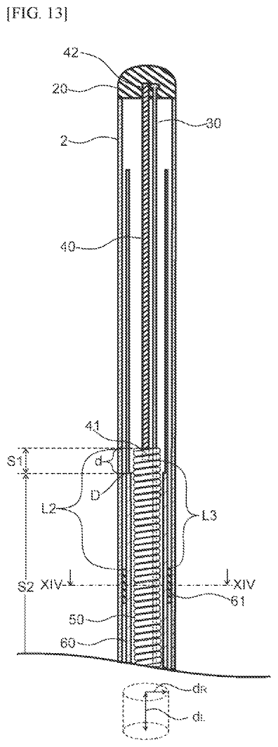

In the second section S2, the inner wall of the tubular member 60 may be in full or partial contact with the outer surface of the coil 50, or may not in contact with the outer surface of the coil 50. As shown in FIG. 13 to FIG. 15, when the inner wall of the tubular member 60 is not in contact with the outer surface of the coil 50, a gap may be formed between the inner wall of the tubular member 60 and the outer surface of the coil 50.

However, when the inner wall of the tubular member 60 is not in contact with the outer surface of the coil 50 in the second section S2, an area s2 of the gap between the inner wall of the tubular member 60 and the outer surface of the coil 50 in the second section S2 in a cross section in the radial direction d_{R} is preferably smaller than an area s1 of the gap between the inner wall of the tubular member 60 and the outer surface of the coil 50 in the first section S1. The tubular member 60 having such a configuration can achieve the above effect. When the inner wall of the tubular member 60 is not in contact with the outer surface of the coil 50 in the second section S2, the location of the position D having the length d proximal to the distal end of the coil means a location where the area s1 between the inner wall of the tubular member 60 and the outer surface of the coil 50 changes to the area s2.

As shown in FIG. 2, FIG. 3, and other figures, preferably, a part of the inner wall of the tubular member 60 is in contact with the outer surface of the coil 50 in the second section S2. More preferably, the entire inner wall of the tubular member 60 is in contact with the outer surface of the coil 50 in the second section S2. This makes it easier to prevent the bulging of the coil 50 in the radial direction d_{R}.

To provide the tubular member 60 so that the inner wall of the tubular member 60 is in contact with the outer surface of the coil 50 in the second section S2, for example, the coil 50 can be placed in the lumen of a resin molded into a tubular shape and the resin can be heat-shrunk outside the coil 50. If the tubular member 60 is formed on the outer periphery of the coil 50 in this manner, the tubular member 60 becomes in close contact with the outer surface of the coil 50 in the second section S2, further easily preventing the coil 50 from bulging in the radial direction da. However, even when the tubular member 60 is formed in such a way so that it adheres closely to the outer surface of the coil 50, the tubular member 60 is not fixed to the outer surface of the coil 50, and the tubular member 60 and the coil 50 may slide against each other in the longitudinal axis direction d_{L}. This improves the flexibility of the catheter 1 because the coil 50 is not bound more than necessary by the tubular member 60. In this case, the first section S1 can be formed by such methods as reducing the thickness of the portion corresponding to the first section S1 of the resin molded into a tubular shape and not heat-shrinking that portion.

The first connection part 41 is preferably located in the first section S1 in the longitudinal axis direction d_{L}. As shown an example in FIG. 5, the first connection part 41 is likely to have high stiffness because it is the portion where the leaf spring 40 is connected to the coil 50, but this portion is located in the first section S1, that is, the inner wall of the tubular member 60 is not in contact with the outer surface of the coil 50 in the portion where the first connection part 41 exists, which prevents further stiffness step due to the tubular member 60.

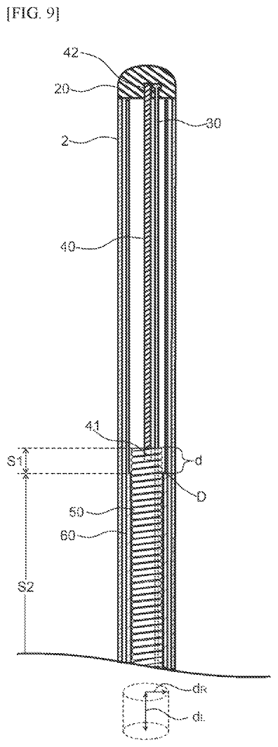

Next, referring to FIG. 9, a catheter in accordance with another embodiment of the present invention will be described. As shown in FIG. 9, preferably, the tubular member 60 extends to the second connection part 42, and the leaf spring 40 and the wire 30 are disposed in the lumen of the tubular member 60 in the distal end part of the shaft 2. Thanks to the tubular member 60 extending to the second connection part 42 in such a way, preferably, the leaf spring 40 and the wire 30 are not exposed in the lumen of the shaft 2. In other words, the leaf spring 40 and the wire 30 are preferably located in the lumen of the tubular member 60. This allows the tubular member 60 to protect the leaf spring 40 and the wire 30, which are easily damaged by repeated bending and traction in repeated use. In addition, in the case where the catheter 1 has a conductive wire connected to an electrode and the like located at the distal end part of the catheter distal part 1D, placing the conductive wire inside the shaft 2 and outside the tubular member 60 can prevent interference of the conductive wire with the leaf spring 40 and the wire 30. The configuration where the tubular member 60 extends to the second connection part 42 includes a configuration where the tubular member 60 extends to the proximal end of the second connection part 42 and a configuration where the tubular member 60 extends to the vicinity of the proximal end of the second connection part 42 in the longitudinal axis direction d_{L}.

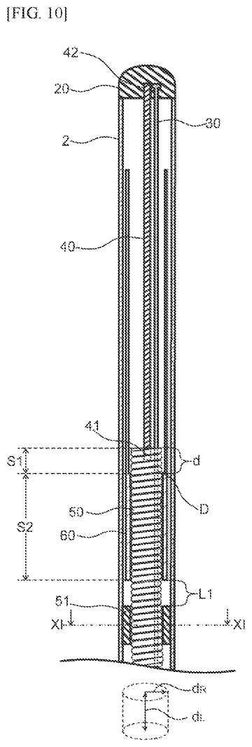

Next, referring to FIG. 10, a catheter in accordance with still another embodiment of the present invention will be described. As shown in FIG. 10, preferably, the proximal end of the tubular member 60 is located distal to the proximal end of the coil 50, and the coil 50 has a first fixed part 51 fixed to the shaft 2 at a position proximal to the proximal end of the tubular member 60 in the longitudinal axis direction d_{L} The coil 50 having the first fixed part 51 fixed to the shaft 2 makes it easier to prevent the twisting of the coil 50 and allows the catheter distal part 1D to bend in a certain direction without kinking. Preferably, the first fixed part 51 is not formed by simply adhering the coil 50 and the shaft 2, but formed by some fixing means, such as adhesive bonding, brazing with solder and the like.

In the longitudinal axis direction d_{L}, a length L1 from a distal end of the first fixed part 51 to the proximal end of the tubular member 60 is preferably a length of 20 windings of the coil 50 or shorter. The length L1 is more preferably a length of 15 windings of the coil 50 or shorter, even more preferably a length of 12 windings or shorter, or may be a length of 10 windings or shorter. In addition, since the first fixed part 51 may be provided in contact with the proximal end of the tubular member 60, an embodiment with the length L1 of 0 (zero), where the distal end of the first fixed part 51 is not apart from the proximal end of the tubular member 60, is also acceptable. Alternatively, the distal end of the first fixed part 51 and the proximal end of the tubular member 60 may be apart from each other, and in this case, the length L1 is preferably a length of 3 windings of the coil 50 or longer, and more preferably a length of 5 windings or longer. With the upper limit of the length L1 in the above range, the first fixed part 51 can be provided near the proximal end of the tubular member 60, which makes it easier to prevent twisting of the coil 50 and to bend the catheter distal part 1D in a certain direction without kinking. With the lower limit of the length L1 in the above range, the first fixed part 51 can be provided at a position proximal to the proximal end of the tubular member 60 by a certain length or longer in the longitudinal axis direction d_{L}, so that the stiffness change due to the tubular member 60 and the stiffness change due to the first fixed part 51 do not overlap, making the flexibility of the catheter distal part 1D improved to more easily achieve the desired curved shape.

Alternatively, although not shown in the figures, the first fixed part 51 may be provided so as to extend to the distal side beyond the proximal end of the tubular member 60 in the longitudinal axis direction d_{L}, and a part of the first fixed part 51 may fix the tubular member 60 and the shaft 2.

The length of the first fixed part 51 in the longitudinal axis direction di, is preferably, for example, a length of 50 windings of the coil 50 or shorter, more preferably a length of 30 windings or shorter, and even more preferably a length of 10 windings or shorter. If the length of the first fixed part 51 in the longitudinal axis direction di, is long, the stiffness of the catheter distal part 1D increases in the area where the first fixed part 51 is provided, which may cause problems such as kinking of the catheter distal part 1D due to the stiffness step. However, the first fixed part 51 having the length in the longitudinal axis direction d_{L} in the above range can reduce the stiffness step caused by the first fixed part 51. In addition, when the catheter 1 is provided with a conductive wire connected to an electrode and other devices located in the tip part, since the conductive wire is disposed inside the shaft 2 and outside the coil 50, the first fixed part 51 having the length in the longitudinal axis direction d_{L} in the above range can reduce the effect of the first fixed part 51 on the conductive wire, preventing the conductive wire from being twitched and allowing the catheter distal part 1D to be easily curved into the desired shape. The lower limit of the length of the first fixed part 51 in the longitudinal axis direction d_{L} is not particularly limited, and for example, it is preferably a length of 3 windings of the coil 50 or longer, more preferably a length of 5 windings or longer, and even more preferably a length of 7 windings or longer. In the longitudinal axis direction d_{L}, preferably, the length of the second section S2, the length of the first fixed part 51, and the length L1 from the distal end of the first fixed part 51 to the proximal end of the tubular member 60 are shortened in this order. Such a configuration can prevent the kinking or deformation such as bulge of the coil 50 thanks to the tubular member 60 and the first fixed part 51, and prevent the effect of stiffness step due to each member, making it easier for the catheter distal part 1D to be curved into the desired curved shape.

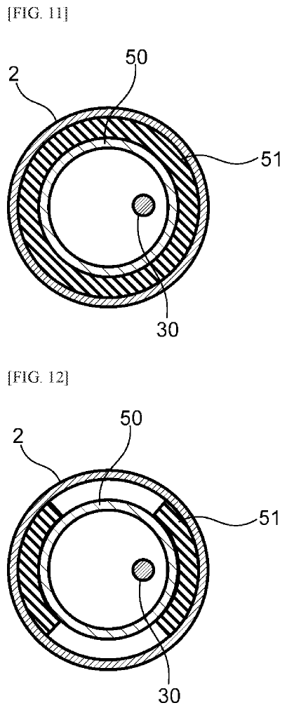

The first fixed part 51 may be provided in the entire range of 360° in the circumferential direction of the space outside the coil 50 and inside the shaft 2 as shown in FIG. 11, or alternatively, may be provided at a part of the range of 360° in the circumferential direction of the space as shown in FIG. 12. When the first fixed part 51 is provided at a part of the range of 360° in the circumferential direction of the space, the number of the first fixed part 51 may be one, or two or more. In this case, the total angle in which the first fixed part 51 is provided in the circumferential direction is preferably 60° or more, and more preferably 90° or more. The first fixed part 51 provided in the above range can secure fixing strength of the coil 50 and the shaft 2. The total angle in which the first fixed part 51 is provided in the circumferential direction is preferably 240° or less, and more preferably 180° or less. The first fixed part 51 provided in the above range can reduce the stiffness step due to the first fixed part 51. In addition, when the catheter 1 is provided with a conductive wire connected to an electrode or other devices located at the tip part, the conductive wire can be inserted in the space outside the coil 50 and inside the shaft 2 at which the first fixed part 51 is not located. This allows the conductive wire to be configured so that it is not fixed by the first fixed part 51, preventing the conductive wire from being twitched and allowing the catheter distal part 1D to be more easily curved into the desired shape.

In one embodiment of the present invention in which the proximal end of the tubular member 60 is located distal to the proximal end of the coil 50 as describe above, the length of the tubular member 60 is ¼ the length of the leaf spring 40 or longer in the longitudinal axis direction d_{L}. This can improve the effect of preventing deformation such as bulge of the coil 50 in the radial direction d_{R} and allow the position of the first fixed part 51 in the longitudinal axis direction d_{L} to be sufficiently separated from the first connection part 41, so that the stiffness change due to the first connection part 41 and the stiffness change due to the first fixed part 51 do not overlap, making it easier to improve the flexibility of the catheter distal part 1D and achieve the desired curved shape.

Next, referring to FIG. 13, a catheter in accordance with still another embodiment of the present invention will be described. As shown in FIG. 13, in the catheter 1 in accordance with still another embodiment of the present invention, the tubular member 60 preferably has a second fixed part 61 fixed to the shaft 2 in the second section S2. The tubular member 60 having the second fixed part 61 fixed to the shaft 2 can prevent the coil 50 from twisting, and thus allowing the catheter distal part 1D to be easily curved in a certain direction without kinking. Preferably, the second fixed part 61 is not formed by simply adhering the tubular member 60 and the shaft 2, but formed by some fixing means, such as adhesive bonding, brazing with solder and the like. The tubular member 60 instead of the coil 50, which has a certain degree of stiffness, is fixed to the shaft 2, thereby preventing kinking of the catheter distal part 1D while improving the flexibility of the catheter distal part 1D. In addition, in an embodiment where the tubular member 60 and the coil 50 can slide against each other in the longitudinal axis direction di, the flexibility of the catheter distal part 1D is unlikely to be lost because a certain degree of freedom is secured in the coil 50 even when the tubular member 60 is fixed to the shaft 2.

In an embodiment like the above, the tubular member 60 may extends to the proximal end of the coil 50 in the longitudinal axis direction d_{L}. Alternatively, the proximal end of the tubular member 60 may be located distal to the proximal end of the coil 50 in the longitudinal axis direction d_{L}, and the coil 50 may further have the first fixed part 51 fixed to the shaft 2 at a position proximal to the proximal end of the tubular member 60. In other words, the catheter 1 may have both the first fixed part 51 and the second fixed part 61.

The length of the second fixed part 61 in the longitudinal axis direction d_{L} is preferably, for example, a length of 50 windings of the coil 50 or shorter, more preferably a length of 30 windings or shorter, and even more preferably a length of 10 windings or shorter. If the length of the second fixed part 61 in the longitudinal axis direction d_{L} is long, the stiffness of the catheter distal part 1D increases in the area where the second fixed part 61 is provided, which may cause problems such as kinking of the catheter distal part 1D due to the stiffness step. However, the second fixed part 61 having the length in the longitudinal axis direction d_{L} in the above range can reduce the stiffness step caused by the second fixed part 61. In addition, when the catheter 1 is provided with a conductive wire connected to an electrode or other devices located at the tip part, since the conductive wire is positioned inside the shaft 2 and outside the coil 50 and the tubular member 60, the second fixed part 61 having the length in the longitudinal axis direction d_{L} in the above range can reduce the effect of the second fixed part 61 on the conductive wire, preventing the conductive wire from being twitched and allowing the catheter distal part 1D to be easily curved into the desired shape. The lower limit of the length of the second fixed part 61 in the longitudinal axis direction d_{L} is not particularly limited, and for example, is preferably a length of 3 windings of the coil 50 or longer, more preferably a length of 5 windings or longer, and even more preferably a length of 7 windings or longer.

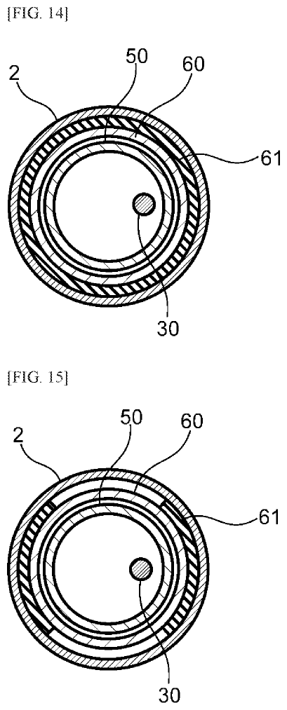

The second fixed part 61 may be provided in the entire range of 360° in the circumferential direction of the space outside the tubular member 60 and inside the shaft 2 as shown in FIG. 14, or alternatively, may be provided at a part of the range of 360° in the circumferential direction of the space as shown in FIG. 15. When the second fixed part 61 is provided at a part of the range of 360° in the circumferential direction of the space, the number of the second fixed part 61 may be one, or two or more. In this case, the total angle in which the second fixed part 61 is provided in the circumferential direction is preferably 60° or more, and more preferably 90° or more. The second fixed part 61 provided in the above range can secure fixing strength of the tubular member 60 and the shaft 2. The total angle in which the second fixed part 61 is provided in the circumferential direction is preferably 240° or less, and more preferably 180° or less. The second fixed part 61 provided in the above range can reduce the stiffness step due to the second fixed part 61. In addition, when the catheter 1 is provided with a conductive wire connected to an electrode or other devices located at the tip part, the conductive wire can be inserted in the space outside the tubular member 60 and inside the shaft 2 at which the second fixed part 61 is not located. This allows the conductive wire to be configured so that it is not fixed by the second fixed part 61, preventing the conductive wire from being twitched and allowing the catheter distal part 1D to be easily curved into the desired shape.

A length L2 from the distal end of the second fixed part 61 to the distal end of the coil 50 in the longitudinal axis direction di, is preferably twice the length of the leaf spring 40 or shorter. The length L2 is more preferably 1.5 times the length of the leaf spring 40 or shorter, and even more preferably one time the length of the leaf spring 40 or shorter. The length L2 is preferably ⅓ the length of the leaf spring 40 or longer, more preferably ½ the length of the leaf spring 40 or longer, and even more preferably ¾ the length of the leaf spring 40 or longer. When the length L2 is in the above range, the tubular member 60 and the shaft 2 can be fixed at a position within a certain distance proximally from the distal end of the coil 50, which is the base end of the curving when the catheter distal part 1D is curved, in the longitudinal axis direction d_{L}, making it easier to prevent the twisting of the coil 50 and allowing the catheter distal part 1D to be curved in a certain direction without kinking.

Preferably, in the longitudinal axis direction d_{L}, the distal end of the second fixed part 61 is located proximal to the proximal end of the first connection part 41, and a length L3 from the distal end of the second fixed part 61 to the proximal end of the first connection part 41 is ¼ the length of the leaf spring 40 or longer. The length L3 is more preferably ⅓ the length of the leaf spring 40 or longer, and even more preferably ½ the length of the leaf spring 40 or longer. The upper limit of the length L3 may be equivalent to the above preferred range of the length L2. The length L3 in the above range allows the position of the second fixed part 61 in the longitudinal axis direction d_{L} to be sufficiently separated from the first connection part 41, so that the stiffness change due to the first connection part 41 and the stiffness change due to the second fixed part 61 do not overlap, making it easier to improve the flexibility of the catheter distal part 1D and achieve the desired curved shape.

The catheter 1 in accordance with still another embodiment of the present invention shown in FIG. 13 may have another fixed part where the coil 50 or the tubular member 60 is fixed to the shaft 2 at a position proximal to the proximal end of the second fixed part 61. If it is more proximal than the proximal end of the second fixed part 61, it is far enough from the distal end of the coil 50 and the first connection part 41 to have little effect on the change in stiffness of the catheter 1. However, the number of another fixed part is preferably the minimum required from the viewpoint of reducing man-hour during manufacturing and, when the catheter 1 has a conductive wire, from the viewpoint of sliding property of the conductive wire. The number of another fixed part in the longitudinal axis direction d_{L} is preferably 5 or less, more preferably 3 or less, and may be 2 or less, 1 or less, or 0.

In the catheter 1 in accordance with still another embodiment of the present invention shown in FIG. 13, preferably, the tubular member 60 and the shaft 2 are not fixed at a position distal to the distal end of the second fixed part 61 in the longitudinal axis direction d_{L}. This can prevent stiffness step in the catheter distal part 1D because no portion near the distal end of the coil 50 is stiffened by the fixed part.

The present application claims priority based on Japanese Patent Application No. 2021-134329 filed on Aug. 19, 2021. All the contents described in Japanese Patent Application No. 2021-134329 filed on Aug. 19, 2021 are incorporated herein by reference.

### DESCRIPTION OF REFERENCE SIGNS

1: catheter
1D: catheter distal part
2: shaft
7: handle
20: tip part
30: wire
40: leaf spring
40a: one surface
40b: the other surface
41: first connection part
42: second connection part
50: coil
51: first fixed part
55: coil wire
60: tubular member
61: second fixed part
70: wire operation part
d: length from the distal end of the coil to the distal end of the tubular member
d_{L}: longitudinal axis direction
d_{R}: radial direction
L: length of the coil in its natural state
Lc: length of the coil at maximum compression
L1: length from the distal end of the first fixed part to the proximal end of the tubular member
L2: length from the distal end of the second fixed part to the distal end of the coil
L3: length from the distal end of the second fixed part to the proximal end of the first connection part
S1: first section
S2: second section

1. A catheter, comprising:
a shaft having a distal end and a proximal end in a longitudinal axis direction and having a lumen extending in the longitudinal axis direction;
a coil having a lumen extending in the longitudinal axis direction and disposed in the lumen of the shaft;
a leaf spring extending in the lumen of the shaft and having a first connection part where a proximal end part of the leaf spring is directly or indirectly connected to a distal end part of the coil and a second connection part where a distal end part of the leaf spring is directly or indirectly connected to a distal end part of the shaft;
at least one wire extending in the lumen of the coil, a distal side of the at least one wire directly or indirectly connected to the distal end part of the shaft; and
a tubular member having a lumen extending in the longitudinal axis direction and disposed outside the coil and inside the shaft such that the at least one wire, the leaf spring, and the first connection part are located in the lumen of the tubular member, wherein
the tubular member and the coil are disposed so that in the longitudinal axis direction, the tubular member has a first section proximally extending from a distal end of the coil to a position D, the first section having a length d, and a second section proximally extending from the position D to a proximal end of the tubular member, an inner wall of the tubular member is not in contact with an outer surface of the coil in the first section, and
the length d is a length of one winding of the coil or longer and a length of 30 windings of the coil or shorter in the longitudinal axis direction.

2. The catheter according to claim 1, wherein a part of the inner wall of the tubular member is in contact with the outer surface of the coil in the second section.

3. The catheter according to claim 1, wherein the first connection part is located in the first section in the longitudinal axis direction.

4. The catheter according to claim 1, wherein the tubular member extends to the second connection part, and the leaf spring and the at least one wire are disposed in the lumen of the tubular member in the distal end part of the shaft.

5. The catheter according to claim 1, wherein the proximal end of the tubular member is located distal to a proximal end of the coil, and the coil has a first fixed part fixed to the shaft at a position proximal to the proximal end of the tubular member in the longitudinal axis direction.

6. The catheter according to claim 5, wherein a length from a distal end of the first fixed part to the proximal end of the tubular member is a length of 20 windings of the coil or shorter in the longitudinal axis direction.

7. The catheter according to claim 1, wherein the tubular member has a second fixed part fixed to the shaft in the second section.

8. The catheter according to claim 7, wherein a length from a distal end of the second fixed part to the distal end of the coil is twice a length of the leaf spring or shorter in the longitudinal axis direction.

9. The catheter according to claim 7, wherein, in the longitudinal axis direction, a distal end of the second fixed part is located proximal to a proximal end of the first connection part, and a length from the distal end of the second fixed part to the proximal end of the first connection part is ¼ a length of the leaf spring or longer.
